(19) United States
(12) Patent Application Publication
(10) Pub. No.: US 2010/0331228 A1
(43) Pub. Date: Dec. 30, 2010

(54) Use of Alkane Sulfonic Acid For Descaling In the Agri-Food Industry

(75) Inventors: Didier Juhue, Chaponost (FR); Bernard Monguillon, Mourenx (FR)

Correspondence Address: SMITH, GAMBRELL & RUSSELL, 1130 CONNECTICUT AVENUE, N.W., SUITE 1130, WASHINGTON, DC, 20036, US

(73) Assignee: ARKEMA FRANCE

(21) Appl. No.: 12/811,398

(22) PCT Filed: Apr. 28, 2009
(86) PCT No.: PCT/FR2009/050786

§ 371 Date: Aug. 27, 2010

(30) Foreign Application Priority Data
Apr. 29, 2008 (FR) 0852879

(51) Int. Cl. C11D 3/60; C07C 309/04; C11D 1/12; C11D 17/00

(52) U.S. Cl. 510/218; 562/30

(57) ABSTRACT

The present invention relates to the use of alkane sulfonic acid for descaling, and more generally for cleaning vats, casks, and other containers used for the storage, shipping, and handling of meat and early produce, particularly poultry, fruit, and vegetables. The present invention also relates to a composition in gel form including at least one alkane sulfonic acid.

The present invention relates to the use of alkanesulfonic acid for the descaling and more generally for the cleaning of vats, casks and other receptacles used for the storage, transportation, handling, preparation or preservation of foodstuffs of plant and animal origins, in particular meat and early horticultural produce, especially fruit and vegetables.

The present invention also relates to a formulation in the gel form comprising at least one alkanesulfonic acid, which formulation is particularly suited to the cleaning and more particularly to the descaling of receptacles, vats, casks, conveyor chains and others contaminated during the storage, handling, transportation and transformation of meat and early horticultural produce, in particular fruit and vegetables.

In the food-processing industry, there exist two particular fields which are that of the chain for distribution of meat, from the abattoirs to the final consumer, and that of the chain for distribution of early horticultural produce, in particular fruit and vegetables, from harvesting to the final consumer.

These two fields, a priori distant, nevertheless both relate to perishable foodstuffs which can generate, by their very nature or during their handling, storage, aging and others, various organic and/or inorganic substances which are deposited and contaminate the various containers, casks, vats, conveyor chains and other receptacles.

This is because the chains for the distribution of meat and early horticultural produce, such as fruit and vegetables, from the producer to the consumer today comprises a great many stages in which the meat and the early horticultural produce are subjected to numerous storage, transportation, sorting, handling, packaging, optionally drying, and other operations.

In each of these stages, use is made of various receptacles, vats, casks, boxes, conveyor chains and others which are in more or less prolonged contact with said foodstuffs, meat and early horticultural produce, such as fruit and vegetables.

As regards contact with foods, the question of cleanliness of the receptacles is thus crucial: the various juices and other solid or liquid impurities can, if they are not efficiently cleaned, result in the formation and the propagation of bacteria and also the formation of scale or any other solid inorganic component which would render said foods unfit for consumption.

Currently, the cleaning in the industry of meat and early horticultural produce is mainly provided by phosphoric acid, in particular due to its high descaling power and its relative corrosiveness with regard to the various receptacles, made of metals or plastics, used in this industry.

However, it is observed that phosphoric acid does not make possible, or only with difficulty, the removal of scale, in particular alkali metal or alkaline earth metal oxalates, which is formed and manages to accumulate in the various receptacles, casks, vats and others. It is in particular very difficult to ensure complete and efficient cleaning which meets the hygiene measures required when the contaminants are present and encrusted in the nooks and crannies or the parts particularly difficult to access of the various items of equipment used for the storage, transportation, handling and others of meat and early horticultural produce.

In addition, for environmental reasons, the use of phosphoric acid is today disputed due to the discharges of phosphate type which it generates.

Thus, a first object of the present invention consists in providing an effective alternative to the use of phosphoric acid for the cleaning of contaminants, in particular contaminants of scale type, more particularly oxalates, encountered in the industry of meat and early horticultural produce, typically meat, fruit and vegetables.

Another object of the present invention consists in providing an effective alternative to the use of phosphoric acid for the efficient cleaning of encrusted contaminants which are difficult to access.

Another object consists in providing an effective alternative to phosphoric acid while avoiding discharges and effluents harmful to the environment.

Yet another object consists in providing an effective alternative to phosphoric acid which does not result in or results in very little corrosion of the various receptacles used for the transportation, handling, transformation and storage, inter alia, of meat, fruit and vegetables.

Yet other advantages will become apparent during the description of the present invention which follows.

It has now been discovered that it is possible to carry out complete and efficient cleaning of the various receptacles used in the industry of meat and early horticultural produce by using a formulation based on alkanesulfonic acid.

This is because it has been discovered that a formulation based on alkanesulfonic acid exhibits a greater descaling power than the phosphoric acid normally used in this type of industry. In addition, the use of an alkanesulfonic acid is less harmful to the environment, in comparison with the discharges of phosphates inherent in the use of phosphoric acid.

Thus, according to a first subject matter, the invention relates to the use of at least one alkanesulfonic acid for the cleaning of the contaminants present in the various receptacles, casks and other containers used in networks for packaging, storing, displaying and distributing the meat resulting from abattoirs and early horticultural produce, in particular fruit and vegetables.

Thus, the method of the invention provides an efficient alternative and one in accordance with environmental regulations to the use of phosphoric acid normally used under these conditions. More specifically, the present invention provides a product for the replacement of phosphoric acid for the cleaning, in particular for the removal, of scale, especially oxalates, said replacement product being biodegradable, less toxic to the environment and more efficient and being able to be used in smaller amounts, while providing a comparable, indeed even better, effectiveness.

More particularly, the use of at least one alkanesulfonic acid according to the invention makes possible the removal of all the types of contaminants encountered in the chain for distribution of meat and early horticultural produce from the producer to the consumer, in particular contaminants such as carbohydrates, fats, proteins, starches or inorganic minerals, such as calcium carbonate, calcium phosphate and other types of scale comprising alkali metal or alkaline earth metal oxalates, sulfates, hydroxides and/or sulfides, in combination or not in combination with various organic and/or metal or semimetal materials.

The use according to the present invention is particularly effective for the removal of scale and in particular alkali metal or alkaline earth metal oxalates and other residues encountered in the field of the packaging, storage, transportation and distribution of meat and early horticultural produce, in particular fruit and vegetables.

Mention may be made, among alkali metal or alkaline earth metal oxalates, preferably and without implied limitation, of sodium oxalate and calcium oxalate and very particularly calcium oxalate.

The term “early horticultural produce” is understood to mean in particular fresh fruit and vegetables which are harvested, stored, transported, handled and put on sale in markets, in hypermarkets, and the like, for the purpose of their consumption. More specifically, the early horticultural produce is fruit and vegetables, and other truck farming produce, among which may be mentioned apples, pears, apricots, cherries, peaches, nectarines, melons, tomatoes, beans, peas, carrots, potatoes, salad vegetables, spinach, endives, squashes, zucchinis, aubergines, turnips, celery, leeks and others, preferably salad vegetables.

The term “meat” is understood to mean, within the meaning of the present invention, meat products generally resulting from abattoirs and which are subsequently cut up, sliced, ground, stored, transported, handled and then put on sale in markets, in hypermarkets, and the like, for the purpose of their consumption. More specifically, the meat products originate from bovines, ovines, caprines, porcines and poultry, more particularly turkeys and chickens.

The use of at least one alkanesulfonic acid is particularly advantageous for the cleaning of the various containers, casks, receptacles, conveying chains and others in more or less prolonged contact with the meat and the vegetables, in particular salad vegetables, lettuces, escaroles and others.

This is because early horticultural produce is subjected to numerous handling operations, from harvesting to the final consumer, and is put into storage for more or less lengthy periods of time in receptacles, containers, casks and other items of equipment which can be composed of various materials, among which may be mentioned stainless steel, aluminum, copper, brass, coated or uncoated steel, for example steel coated with an epoxy resin, plastic, in particular polypropylene, polyethylene or poly(vinyl chloride), glass and others.

Upon more or less prolonged contact with meat and early horticultural produce, the receptacles, casks and other items of equipment are contaminated by the various types of abovementioned contaminations, which it is advisable to remove for obvious reasons of food hygiene and safety, as indicated above.

In the present invention, the term “alkanesulfonic acid” is understood to mean preferably the alkanesulfonic acids comprising a saturated and linear or branched hydrocarbon chain comprising from 1 to 4 carbon atoms.

The alkanesulfonic acids which can be used in the context of the present invention are chosen in particular from methanesulfonic acid, ethanesulfonic acid, n-propanesulfonic acid, isopropanesulfonic acid, n-butanesulfonic acid, isobutanesulfonic acid, sec-butanesulfonic acid, tert-butanesulfonic acid and the mixtures of two or more of them in all proportions.

According to a preferred embodiment, the alkanesulfonic acid used in the context of the present invention is methanesulfonic acid or ethanesulfonic acid; entirely preferably, the acid used is methanesulfonic acid.

Thus, the use according to the present invention employs at least one alkanesulfonic acid chosen from alkanesulfonic acids having a linear or branched chain comprising from 1 to 4 carbon atoms and preferably at least methanesulfonic acid (MSA).

Any type of formulation comprising at least one alkanesulfonic acid may be suitable. As a general rule, the formulation comprises from 0.1 to 100% by weight of alkanesulfonic acid, more generally from 0.5 to 90% by weight, in particular from 0.5 to 50% by weight and more particularly from 0.5 to 35% by weight of alkane-sulfonic acid.

The formulation is, for example, an aqueous formulation which can be prepared in the form of a concentrated mixture which is diluted by the final user. In an alternative form, the formulation can also be a ready-for-use formulation, that is to say that it does not have to be diluted. Use may be made, for example, of the methanesulfonic acid in aqueous solution sold by Arkema under the name Scaleva™, ready-for-use or diluted with water in the proportions shown above.

According to an entirely preferred alternative form, the formulation is a formulation in the gel form. This is because it has been observed that the formulations in the gel form of alkanesulfonic acid(s) are very effective for industrial cleaning, not only due to the gel itself, which makes possible a longer action of the acid active principle (the gel “adheres” for a longer time to the surfaces, in comparison with an aqueous formulation), but also exhibits an improved cleaning power, in comparison with other gel formulations, for example formulations in the phosphoric acid gel form.

The improved cleaning power of a formulation in the gel form of at least one alkanesulfonic acid is particularly noteworthy in the case of descaling, in particular for the removal of oxalates, such as those which may be encountered in the fields of meat and early horticultural produce, more particularly for the removal of contaminants of oxalate type present in the various receptacles, casks and other containers used for the transformation, packaging, storage and distribution of meat and early horticultural produce, in particular fruit and vegetables, very particularly turkeys, poultry and salad vegetables.

Thus, according to another aspect, the present invention relates to a formulation in the gel form comprising:
from 0.5 to 90% by weight, preferably from 0.5 to 50% by weight and more particularly from 0.5 to 35% by weight of at least one alkanesulfonic acid, preferably methanesulfonic acid;
from 0.1 to 20% by weight, preferably from 0.5 to 10% by weight and more particularly from 1 to 8% by weight of at least one gelling agent;
from 0.1 to 20% by weight, preferably from 0.5 to 10% by weight, of at least one surfactant; and
the remainder to 100% of water.

The gelling agents and the surfactants which can be used in the formulations in the gel form of the present invention can be of any type. A person skilled in the art will know how, without particular difficulty and drawing inspiration from the following examples, to choose and adapt the nature of the gelling agents and surfactants which are appropriate. Some surfactants can act as gelling agents and, in this case, the addition of a specific gelling agent is not necessary. Likewise, some gelling agents can act as surfactant, rendering the addition of a specific surfactant unnecessary.

According to another aspect, the present invention relates to the formulations in the foaming gel form. This is because foaming gels are very particularly advantageous due to the fact that they produce a clinging foam, in other words an adherent foam, at the walls of the various items of equipment and devices to be cleaned, while requiring a reduced consumption of cleaning acid active material, and exhibit the advantage of a better ability to be rinsed off, that is to say simpler and more efficient removal, while requiring a smaller amount of water.

Thus, the present invention relates to a formulation in the foaming gel form comprising:
from 0.5 to 90% by weight, preferably from 0.5 to 50% by weight and more particularly from 0.5 to 35% by weight of at least one alkanesulfonic acid, preferably methanesulfonic acid;
from 0.1 to 20% by weight, preferably from 0.5 to 10% by weight and more particularly from 1 to 8% by weight of at least one foaming agent;
from 0 to 20% by weight, preferably from 0.5 to 10% by weight and more particularly from 1 to 8% by weight of at least one gelling agent;
from 0 to 10% by weight, preferably from 0.1 to 5% by weight, of a solubilizing or hydrotropic agent;
from 0 to 20% by weight, preferably from 0.5 to 10% by weight, of at least one surfactant; and
the remainder to 100% of water.

Depending on the field and the method of application, the formulation can be prepared in the concentrate form and thus with a low appropriate viscosity and can then be diluted before use until the expected effectiveness with regard to the viscosity and the foaming power is obtained.

In the foaming gel formulation according to the invention, the gelling agents and surfactants are generally of the same type as the gelling agents and surfactants used for the formulations in the gel form described above, that is to say of any type known to the person skilled in the art who will know how, without particular difficulty and drawing inspiration from the following examples, to choose and adapt the nature of the gelling agents and surfactants which are appropriate. As indicated above, some surfactants can act as gelling agents and, in this case, the addition of a specific gelling agent is not necessary. Likewise, some gelling agents can act as surfactant, rendering the addition of a specific surfactant unnecessary.

In the above foaming gel formulation, the foaming agent can be chosen from the foaming agents commonly used by a person skilled in the art and preferably from amine oxides, such as, for example:

dimethylalkylamine oxides, the alkyl chain being a “fatty” chain comprising, for example, from 10 to 30 carbon atoms, preferably from 12 to 22 carbon atoms, for example Empigen® OB from Huntsman,

ethoxylated amine oxides, and

mixtures of two or more of them.

The use of at least one ethoxylated amine oxide, such as, without implied limitation, Cecajel® OX100 from Ceca or Aromox® T12 from Akzo, alone or in combination with at least one dimethylalkylamine oxide, makes it possible to contribute stability to the foaming gel.

Foaming agents, in particular those described above, generally form gels when they are mixed with water, that is to say that they increase the viscosity of the formulation without it being necessary to add a gelling agent. However, the addition of such a gelling agent is not excluded from the present invention.

Mention may be made, among solubilizing or hydrotropic agents which can be used in the formulations according to the invention, by way of example and without implied limitation, of sodium xylene- or cumenesulfonates. However, such agents are not essential in the acid formulations according to the invention.

The formulations in the foaming gel form can be applied according to any method known to a person skilled in the art, and in particular under pressure, or else using a spray gun.

The aqueous formulations in the gel or foaming gel form of the present invention can also comprise one or more additives, solvents and other rheological or texturizing agents chosen from solvents and cosolvents (preferably water-soluble solvents and cosolvents, for example alcohols, such as ethanol, isopropanol and others), organic or inorganic acids (for example hydrochloric, sulfuric, phosphoric, nitric, amidosulfonic, sulfamic, citric or formic acid), salts of organic or inorganic acids, in particular alkali metal or alkaline earth metal salts (for example chlorides, sulfates, phosphates or nitrates, in particular sodium chloride), thickening agents, surfactants, foaming agents, antifoaming agents, flame retardants, preservatives, disinfectants (for example chosen, without implied limitation, from peracetic acid, bromoacetic acid, and others), fragrances, colorants and others known to a person skilled in the art.

An aqueous formulation or a formulation in the gel form or also a formulation in the foaming gel form which is particularly preferred is a formulation comprising from 0.5 to 50% by weight, preferably from to 35% by weight, preferably approximately 20% by weight, of methanesulfonic acid.

The cleaning of the various receptacles, casks, conveying chains and other containers or items of equipment in more or less prolonged contact with the meat or early horticultural produce, as indicated above, is carried out by contact (immersion, sprinkling, spraying) with an effective amount of at least one alkanesulfonic acid, preferably methanesulfonic acid, in the form of aqueous formulations, preferably of an aqueous gel or of a foaming gel, as have just been described.

This cleaning operation can be repeated one or more times according to the amount of contaminants to be removed and their degrees of encrustation. However, the use of at least one alkanesulfonic acid, preferably methanesulfonic acid, requires only a single cleaning stage, in particular for the removal of scale, especially oxalates.

This has proved to be particularly true when said at least one alkanesulfonic acid is used in the aqueous gel form, better still in the aqueous foaming gel form. Very good results have, however, been observed with aqueous liquid formulations comprising at least one alkanesulfonic acid.

The cleaning stage or stages described above can, if appropriate, comprise, be supplemented by or be followed by one or more disinfecting stages, disinfecting being carried out according to conventional techniques known to a person skilled in the art, using disinfecting compounds, biocides or any other compound suitable for such a use.

The cleaning and optionally disinfecting stage or stages can optionally, but preferably, be followed by one or more rinsing operations, preferably with clear water.

In the method of the invention as just described, the term “effective amount” is understood to mean an amount which makes it possible to remove all the contaminants which, if they are not correctly removed, might promote the growth of bacteria.

This amount can vary within wide limits, according to the volume and the number of items of equipment to be cleaned, the nature and the amount of contaminants which it is desired to remove, the temperature and the pressure of the formulation used, the desired duration of the cleaning and others.

A visual test of the various items of equipment cleaned, according to conventional techniques known to a person skilled in the art, makes it possible to determine the effective amount of formulation to be used, the temperature and the duration of cleaning necessary for the partial or complete removal of the contaminants.

Thus, the amount of acid will advantageously be established in order to make possible complete removal of the contaminants, while observing a minimum amount of acid, essentially for economic reasons.

Alkanesulfonic acids, in particular alkane-sulfonic acid, exhibit a greater descaling power than phosphoric acid, which makes it possible to use smaller amounts of acids which are more respectful of the environment: the effluents (residues, salts and others) resulting from a cleaning operation using at least one alkanesulfonic acid are biodegradable, in contrast to the phosphoric acid commonly used today, which generates phosphates, responsible, inter alia, for phenomena of eutrophication of rivers, watercourses and reserves of water, such as lakes, marshes, water tables, and the like.

The acid cleaning with at least one alkane-sulfonic acid as indicated above can be carried out at any temperature, generally of between 0° C. and 150° C., more generally of between 5° C. and 100° C., typically between 10° C. and 80° C.

This cleaning with at least one alkanesulfonic acid is generally followed by one or more rinsing operations, for example with clear water, in a way known to a person skilled in the art. Depending on the presence or not in the formulation of a disinfecting agent, an additional stage of bacteriological disinfection may take place according to conventional techniques known to a person skilled in the art.

By virtue of the use of the present invention, the cleaning of the various items of equipment, receptacles, casks and other containers used in the industry of early horticultural produce and meat makes it possible to remove in particular contaminations of scale type, more particularly oxalates.

The present invention is illustrated by means of the examples which follow, without exhibiting any limiting nature, and which cannot consequently be understood as capable of restricting the scope of the invention as claimed.

### EXAMPLE 1

### Preparation of a Methanesulfonic Acid Gel

A gel comprising 10% by weight of methanesulfonic acid is prepared by successively adding, with stirring, the water, the methanesulfonic acid, the surfactant and the gelling agent in the following proportions:

| | | |
|---|---|---|
| Water: | 160.4 | g |
| Methanesulfonic acid (70% in water, Arkema) | 28.6 | g |
| Gelling agent (Cecajel ® 210, Ceca) | 8.0 | g |
| Surfactant (Remcopal ® LM4F, Arkema) | 3.0 | g |

The gel obtained exhibits a clear and non-runny appearance. The viscosity is measured on a Brookfield DV II+device. All the measurements are carried out at ambient temperature. A viscosity of 240 cP is thus measured at 10 revolutions per minute and a viscosity of 230 cP is thus measured at 30 revolutions per minute.

The gel obtained is stable, without observation of phase separation after 2 months at 40° C.

### EXAMPLE 2

### Preparation of a Phosphoric Acid Gel

### Comparative Example

A gel comprising 10% by weight of phosphoric acid is prepared by successively adding, with stirring, the water, the phosphoric acid, the surfactant, the gelling agent and the fragrance in the following proportions:

| | | |
|---|---|---|
| Water: | 174.4 | g |
| Phosphoric acid (85%) | 21.6 | g |
| Gelling agent (Cecajel ® 210, Ceca) | 3 | g |
| Surfactant (NaXS: 40% sodium | 1 | g |
| xylenesulfonate, Huntsman) | | |

The gel obtained exhibits a clear and non-runny appearance. The viscosity is measured as above and is 450 cP at 10 revolutions per minute and 440 cP at 30 revolutions per minute.

The gel obtained is stable, without observation of phase separation after 2 months at 40° C.

### EXAMPLE 3

### Descaling Power

The descaling power is determined by applying the following protocol:
a sheet of marble with the dimensions 10×75×150 mm, exhibiting a polished face, is rinsed in ethanol and rubbed with a brush;
the sheet is subsequently rinsed with running water while rubbing with a brush;
the sheet is dried in an oven at 110° C. for at least 1 hour;
the sheet is then placed in a desiccator in order for it to cool without taking up moisture again;
the sheet is weighed to within an accuracy of 0.01 g with a balance;
the sheet is subsequently entirely immersed in the test gel and is left to soak for 10 s;
the sheet is removed from the gel and suspended while being allowed to drain for 10 minutes;
the sheet is rinsed with running water while rubbing with a brush;
the sheet is dried at 110° C. for at least 1 hour and is then placed in a desiccator in order to cool without moisture;
the sheet is weighed to within an accuracy of 0.01 g on the same balance;
the difference in the weights (Δw) corresponds to the scale destroyed by the gel.

The results of the characterization of the MSA and phosphoric acid gels are compiled in the following table 1:

TABLE 1

| Gel formulation | Δw (g) |
|---|---|
| Example 1 (MSA) | 310 |
| Example 2 (phosphoric acid) | 250 |

The descaling power of the methanesulfonic acid, formulated in the gel form at 10% by weight, is greater than the descaling power of the phosphoric acid, formulated in the gel form at 10% by weight.

Methanesulfonic acid is thus more effective, in particular when it is used in the gel form, than phosphoric acid for descaling operations.

In addition to this advantage, methanesulfonic acid is biodegradable, which makes it a replacement product of choice for phosphoric acid for cleaning operations, in particular descaling operations, more particularly when formulated in the gel form.

### EXAMPLE 4

### Tests on Viscosity and Stability of Gel Formulations

The gel formulations 4a to 4g are prepared according to example 1. Their compositions by weight, expressed in grams, are presented in the following table 2:

TABLE 2

| | Ex. 4a | Ex. 4b | Ex. 4c | Ex. 4d | Ex. 4e | Ex. 4f | Ex. 4g |
|---|---|---|---|---|---|---|---|
| Water | 88.86 | 81.1 | 82.1 | 70.58 | 72.58 | 61.43 | 64.43 |
| Scaleva ™* | 7.14 | 12.9 | 12.9 | 21.42 | 21.42 | 28.57 | 28.57 |
| Cecajel 210** | 2.5 | 3 | 2.5 | 4 | 3 | 5 | 3.5 |
| NaCl | 2.5 | 3 | 2.5 | 4 | 3 | 5 | 3.5 |
| Content by weight of | 5% | 9% | 9% | 15% | 15% | 20% | 20% |
| active material (MSA) | | | | | | | |

*Scaleva ™: 70% by weight aqueous methanesulfonic acid solution.
**The Cecajel 210, gelling agent, also acts as surfactant.

The viscosities of the gels 4a to 4g are measured, according to the method described in example (dynamic viscosity measured in centipoises (cP) at 12 and 30 revolutions per minute (rpm) on a Brookfield DV II+device at ambient temperature), immediately after the preparation of the formulation and then after storage for 2 months at ambient temperature. The results are presented in the following table 3:

TABLE 3

| Brookfield viscosity | | Ex. 4a | Ex. 4b | Ex. 4c | Ex. 4d | Ex. 4e | Ex. 4f | Ex. 4g |
|---|---|---|---|---|---|---|---|---|
| After | 12 rpm | 515 | 820 | 270 | 780 | 320 | 715 | 320 |
| preparation | 30 rpm | 495 | 800 | 270 | 775 | 320 | 710 | 320 |
| After 2 | 12 rpm | 500 | 907.5 | 382.5 | 1007.5 | 482.5 | 1000 | 425 |
| months | 30 rpm | 480 | 890 | 370 | 1000 | 480 | 1000 | 420 |

The above values show, on the one hand, that it is possible to prepare formulations in the alkane-sulfonic acid gel form at various concentrations of active material and, on the other hand, that these formulations in the gel form are stable over time.

### EXAMPLE 5

### Methanesulfonic Acid Formulations in the Foaming Gel Form

The foaming gel formulations 5a and 5b are prepared in a similar way to that employed for the preparation of the gels. The compositions by weight, expressed in grams, are presented in the following table 4:

TABLE 4

| | Ex. 5a | | Ex. 5b |
|---|---|---|---|
| Water | 81.1 | Water | 82.8 |
| Cecajel 210 | 2.5 | Cecajel 210 | 2.5 |
| Cecajel OX100 | 1.0 | Empigen OB | 1.0 |
| NaCl | 2.5 | NaXS | 0.8 |
| Scaleva ™* | 12.9 | Scaleva ™* | 12.9 |

*Scaleva ™: 70% by weight aqueous methanesulfonic acid solution.

The viscosities of the gels 5a and 5b are measured, according to the method described in example 1 (dynamic viscosity measured in centipoises (cP) on a Brookfield DV II+device at ambient temperature), immediately after the preparation of the formulation. The results are presented in the following table 5:

TABLE 5

| Brookfield viscosity | Ex. 5a | Brookfield viscosity | Ex. 5b |
|---|---|---|---|
| 6 rpm | 3550 | 6 rpm | 1500 |
| 3 rpm | 3600 | 12 rpm | 1500 |

The above values show, on the one hand, that it is possible to prepare formulations in the alkane-sulfonic acid foaming gel form at various concentrations of active material. In addition, it has been observed that these formulations in the foaming gel form are stable over time.

### EXAMPLE 5

### Cleaning Tests on Meat Trays

A piece of beef (approximately 1 kg) is left for three days in a rectangular tray (aluminum) in a chamber placed at +3° C. at atmospheric pressure (cold room). After the three days, the piece of meat is removed from the tray. The tray comprises various organic and inorganic contaminants, including calcium oxalate, determined by infrared spectrophotometry. The test is repeated 3 times.

Each tray is then cleaned by bringing the contaminants into contact with 250 ml of a cleaning formulation. Each tray is subsequently rinsed twice with 1 liter of water at each rinsing operation.

The cleaning quality is evaluated visually and recorded according to the following criteria:

0: no cleaning
+: numerous remains of contaminants
++: a few remains of contaminants
+++: traces of contaminants
++++: no trace of contaminants

The results are presented in the following table 6:

TABLE 6

| Formulation | Cleaning quality |
|---|---|
| Formulation of example 1 (MSA gel) | ++++ |
| Formulation of example 2 (comparative) | +++ |
| Liquid formulation (liquid solution | +++ |
| comprising 10% by weight of MSA in water) | |

### EXAMPLE 6

### Cleaning Tests on Vegetable Trays

A large amount of spinach (approximately 3 kg), an item rich in calcium oxalate, is introduced into an aluminum water vat where it is stirred with a to-and-fro movement for several minutes at ambient temperature. The water is subsequently removed and the tray comprises various insoluble impurities, including calcium oxalate, determined by infrared spectro-photometry. The test is repeated twice.

Each tray is then cleaned by bringing the contaminants into contact with 250 ml of a cleaning formulation. Each tray is subsequently rinsed twice with 1 liter of water in each rinsing operation.

The cleaning quality is evaluated visually and recorded according to the same criteria as those defined in example 6. The results are presented in the following table 7:

TABLE 7

| Formulation | Cleaning quality |
|---|---|
| Formulation of example 1 (MSA gel) | ++++ |
| Formulation of example 2 (comparative) | +++ |
| Liquid formulation (liquid solution | ++++ |
| comprising 10% by weight of MSA in water) | |

The two preceding examples show that the formulations based on alkanesulfonic acid, whether liquid or in the gel form, exhibit cleaning properties entirely comparable with, indeed even better than, those observed for a phosphoric acid gel formulation. These formulations based on alkanesulfonic acid, in particular based on methanesulfonic acid, exhibit in addition the advantage of being biodegradable and more respectful of the environment, in comparison with the formulations based on phosphoric acid.

1. The use of at least one alkanesulfonic acid for the cleaning of the contaminants present in the various receptacles, casks and other containers used in networks for packaging, storing, displaying and distributing in the field of meat and early horticultural produce, in particular poultry, fruit and vegetables, said contaminants being of scale type, in particular alkali metal or alkaline earth metal oxalates.

2. The use as claimed in claim 1, in which the early horticultural produce is fruit and vegetables, and other truck farming produce, including apples, pears, apricots, cherries, peaches, nectarines, melons, tomatoes, beans, peas, carrots, potatoes, salad vegetables, spinach, endives, squashes, zucchinis, aubergines, turnips, celery, leeks and others, preferably salad vegetables.

3. The use as claimed in claim 1, in which the field of meat comprises meat products originating from bovines, ovines, caprines, porcines and poultry, more particularly turkeys and chickens.

4. The use as claimed in claim 1, in which the alkanesulfonic acid is chosen from methanesulfonic acid, ethanesulfonic acid, n-propanesulfonic acid, isopropanesulfonic acid, n-butanesulfonic acid, isobutanesulfonic acid, sec-butanesulfonic acid, tert-butanesulfonic acid and the mixtures of two or more of them in all proportions.

5. The use as claimed in claim 1, in which the alkanesulfonic acid is methanesulfonic acid or ethanesulfonic acid, preferably methanesulfonic acid.

6. The use as claimed in claim 1, in which the alkanesulfonic acid is used in a formulation comprising from 0.1 to 100% by weight, more generally from 0.5 to 90% by weight, in particular from 0.5 to 50% by weight and more particularly from 0.5 to 35% by weight of alkanesulfonic acid.

7. A formulation in the gel form comprising:
from 0.5 to 90% by weight, preferably from 0.5 to 50% by weight and more particularly from 0.5 to 35% by weight of at least one alkanesulfonic acid, preferably methanesulfonic acid;
from 0.1 to 20% by weight, preferably from 0.5 to 10% by weight and more particularly from 1 to 8% by weight of at least one gelling agent;
from 0.1 to 20% by weight, preferably from 0.5 to 10% by weight, of at least one surfactant; and
the remainder to 100% of water.

8. A formulation in the foaming gel form comprising:
from 0.5 to 90% by weight, preferably from 0.5 to 50% by weight and more particularly from 0.5 to 35% by weight of at least one alkanesulfonic acid, preferably methanesulfonic acid;
from 0.1 to 20% by weight, preferably from 0.5 to 10% by weight and more particularly from 1 to 8% by weight of at least one foaming agent;
from 0 to 20% by weight, preferably from 0.5 to 10% by weight and more particularly from 1 to 8% by weight of at least one gelling agent;
from 0 to 10% by weight, preferably from 0.1 to 5% by weight, of a solubilizing or hydrotropic agent;
from 0 to 20% by weight, preferably from 0.5 to 10% by weight, of at least one surfactant; and
the remainder to 100% of water.

9. The formulation as claimed in claim 7, in which the alkanesulfonic acid is methanesulfonic acid.

10. The formulation as claimed in claim 7, comprising from 0.5 to 50% by weight, preferably from 10 to 35% by weight, preferably approximately 20% by weight, of methanesulfonic acid.

11. The formulation as claimed in claim 7, additionally comprising one or more additives, solvents and other rheological or texturizing agents chosen from solvents and cosolvents, organic or inorganic acids, salts of organic or inorganic acids, thickening agents, surfactants, foaming agents, antifoaming agents, flame retardants, preservatives, disinfectants, fragrances, colorants and others.

12. The formulation as claimed in claim 8, in which the alkanesulfonic acid is methanesulfonic acid.

13. The formulation as claimed in claim 8, comprising from 0.5 to 50% by weight, preferably from 10 to 35% by weight, preferably approximately 20% by weight, of methanesulfonic acid.

14. The formulation as claimed in claim 8, additionally comprising one or more additives, solvents and other rheological or texturizing agents chosen from solvents and cosolvents, organic or inorganic acids, salts of organic or inorganic acids, thickening agents, surfactants, foaming agents, antifoaming agents, flame retardants, preservatives, disinfectants, fragrances, colorants and others.
